(19) United States
(12) Patent Application Publication
(10) Pub. No.: US 2004/0018576 A1
(43) Pub. Date: Jan. 29, 2004

(54) Bence Jones protein testing cassette

(76) Inventors: Todd M. DeMatteo, Farmington, CT (US); John Liseo, Cromwell, CT (US)

Correspondence Address: ST. ONGE STEWARD JOHNSTON & REENS, LLC, 986 BEDFORD STREET, STAMFORD, CT, 06905-5619, US

(21) Appl. No.: 10/202,587

(22) Filed: Jul. 24, 2002

(51) Int. Cl. G01N 33/53; G01N 33/537; G01N 33/543; C12M 1/34

(52) U.S. Cl. 435/7.93; 435/287.2

(57) ABSTRACT

An analytical device for the determination of the presence of free light chains (Bence Jones protein), and classes thereof (kappa/lambda) in a unconcentrated and undiluted urine sample is provided in which the sample is reacted with labeled binding reagent. The presence of the free light chains is revealed by improved specific binding assay methods, including kits and devices utilizing chromatographically mobile specific binding reagents labeled with colloidal particles. Specific binding reagents labeled with colloidal particles such as gold may be subjected to rapid chromatographic solvent transport on chromatographic media by means of selected solvents and chromatographic transport facilitating agents.

### FIELD OF THE INVENTION

The present invention relates generally to assay devices and specifically to those devices making use of chromatographic techniques in conducting specific binding assays for the determination of the presence of light chains (free & bound) in human urine. Test kits for kappa, and lambda are demonstrated. The invention relates to Bence Jones test kits suitable for use at home, or in a doctor's office, which are intended to give an analytical result rapidly and which require minimum degree of skill and involvement from the user.

### BACKGROUND OF THE INVENTION

The use of reagent-impregnated test strips in specific binding assays, such as immunoassays, has previously been proposed. In such procedures a sample is applied to one portion of the test strip and is allowed to permeate through the strip material, usually with the aid of an eluting solvent such as water. In so doing, the sample progresses into or through a detection zone in the test strip wherein a specific binding reagent is immobilized. Analyte present in the sample can participate in a sandwich or a competition reaction within the detection zone, with a labeled reagent which can also be incorporated in the test strip or applied thereto.

Tests for determining the presence of light chains (free and bound) are also presently known in the art, however the use of such chromatographic strips for the detection of light chains however has not been heretofore proposed or used for the detection of light chains in untreated urine as described herein. This invention solves the problems of contamination risk, insensitivity, unreliability, high cost, and time consumption of the typical state of the art tests, as presently known.

The present invention provides an improved, less expensive, easy to use test for the detection of free and bound light chains, as well as simplified test procedures without the prior problems of known tests which can be laborious, time consuming, costly, and require skill in interpretation. The chromatographic method and devices of the present invention is capable of reliably sensing free and bound light chains with increased sensitivity and require minimal interpretation. The improved method and devices of the present invention enable physicians and assistants thereof the ability to routinely perform such a test for the detection of free light chains in a doctor's office without the wasted time and expense of sending urine samples to a lab for laborious examination. In cases where the labor-intensive state of the art tests have been performed, the claimed invention can also be used to quickly verify any results.

It is known that an immunoglobulin is schematically made up of two heavy chains and two light chains. Determinations of the presence of the free light chains, also called Bence Jones proteins, which pass into the urine is of great interest from a diagnostic viewpoint.

Gammopathies characterized by the proliferation of B cell clones, yield increased clonal production of immunochemicals. In addition to the primary effects of the gammopathy, secondary consequences dependent on the class of clonal immunochemical produced may emerge. The preferential production of single immunoglobulin clones will result in immunodefiency as a result of normal immunoglobulin production, and the production of toxic free light chains will cause renal disease even at low-level depositation. Immunological pathologies caused by the presence of free light chains in the urine, which is the consequence of the increase thereof in the blood, is associated with and can be summarized as (a) the presence of monoclonal free light chains, i.e. immunoproliferative illnesses such as multiple myeloma, micromolecular myeloma, Waldenstrom's macroglobulinemia, chronic lymphatic leukemia and primitive amyloidosis; and (b) the presence of polyclonal free light chains, i.e. hyperimmune illnesses such as systemic lupus erythematosus, acute rheumatoid arthritis and secondary amyloidosis.

The presence of free light chains in urine presupposes their anomalous increase in the serum of the subject but, given their low molecular weight, free light chains pass through the glomerular filter and do not persist in the blood. It is therefore necessary to perform an indirect investigation, ascertaining their presence in the urine.

Known diagnostic methods based on ascertainment of free light chains in the urine are of great interest but at present are blocked by the difficulties of performance of such an investigation. Current techniques such as that described in U.S. Pat. No. 5,141,877 to Massaro, herein incorporated by reference, necessarily require concentration of the sample because of the relatively small percentage of free light chains in the organic liquid even with serious pathological conditions of the subject. Electrophoretic examination of the unconcentrated sample results in unacceptably low sensitivity and the resulting unreliability. The time necessary for concentration is added however to the time required for electrophoretic analysis with the obvious drawbacks. The analysis performed on the concentrated samples undoubtedly raises the reliability of the results without however achieving reasonable certainty. On the samples which prove suspect under electrophoresis it is therefore very advisable to perform immunofixation or immunoelectrophoretic tests, the laboriousness and cost, and requirement for interpretation of which are known, to achieve truly satisfactory levels of sensitivity and hence reliability of the analysis results.

U.S. Pat. No. 5,141,877 describes a method for the determination of the presence of free light chains (Bence Jones protein) in a urine sample in which the sample is reacted with an anti-free light chain antiserum reagent, where the presence of the free light chains is revealed by increase in turbidity of the reacted sample. By comparison with the turbidity of calibrators having predetermined concentrations reacted with anti-free light chain antiserum, a quantitative analysis of the amount of free light chains in the urine sample can be determined. The test samples are derived from patients having secretory micromolecular myeloma.

Of interest to the present application is the disclosure of U.S. Pat. No. 4,727,037, issued to Ring, relating to a method for determining the class and subclass of a test antibody which demonstrates adsorbing on nitrocellulose paper or diazo paper in a predetermining spatial arrangement, antisera directed to isotype class and subclasses of an immunoglobulin; contacting the adsorbed isotype specific antisera with a fluid containing a test antibody to form a complex between the specific immunoglobulin heavy and light chains of the adsorbed antisera and the test antibody present in the fluid; and reacting the complex so formed with a chromogenic substrate to thereby identify the specific class or subclass of the test antibody.

U.S. Pat. No. 5,569,608 describes a method for determining the concentration of analyte in a test fluid by immunochromatography techniques which involves quantitatively determining the signals from captured analyte/labeled binding partner complex by an instrument, e.g. a reflectance spectrometer. A reflectance reading is determined for the captured complex and uncomplexed labeled binding partner which is captured in a separate zone of the immunochromatographic strip and the ratio of these reflectances is used to provide additional quantification to the assay method.

U.S. Pat. No. 5,780,308 teaches a multizone test device for semiquantitatively determining the presence of at least a predetermined minimum concentration of an analyte in a test sample. The device utilizes a strip of porous material, the strip comprising a reagent zone and a capture site. The reagent zone is upstream from the capture site. The reagent further contains a soluble conjugate comprising a labeled analyte-specific binding member which binds the analyte to form a labeled analyte complex. In addition the reagent zone has a capture reagent comprising an unlabeled specific binding member attached to the porous material. The capture reagent binds the labeled analyte complex to form an immobilized labeled analyte complex. Further the reagent zone includes a soluble calibration reagent comprising an unlabeled specific binding member which blocks the binding of the analyte to the capture reagent, thereby controlling the proportion of the analyte that binds to the capture reagent. As a consequence the analyte in the test sample must exceed a minimum concentration before the immobilized labeled complex is formed. The calibration reagent is contained in the reagent zone. The capture reagent is immobilized at the capture site where the immobilized labeled complex is separated from the test sample, and where the presence of label associated with the immobilized labeled complex is detected to determine the presence of at least a predetermined minimum concentration of an analyte in the test sample.

U.S. Pat. No. 5,989,921, which is herein incorporated by reference relates to a test cell and a method for detection of a preselected ligand in a liquid sample such as a body fluid. The test cell includes an elongate outer casing which houses an interior permeable material capable of transporting an aqueous solution and defining a sample inlet, a test volume, and a reservoir volume. The reservoir volume is disposed in a section of the test cell spaced apart from the inlet and is filled with sorbent material. The reservoir acts to receive liquid transported along a flow path defined by the permeable material and extending from the inlet and through the test volume.

Also of interest is U.S. Pat. No. 6,352,862 to Davies et al., which is herein incorporated by reference, which relates to an analytical test device incorporating a dry porous carrier to which a liquid sample, e.g. urine, suspected of containing an analyte such as HCG or LH can be applied indirectly, the device also incorporating a labeled specific binding reagent which is freely mobile in the porous carrier when in the moist state, and an unlabeled specific binding reagent which is permanently immobilized in a detection zone on the carrier material, the labeled and unlabeled specific binding reagents being capable of participating in either a sandwich reaction or a competition reaction in the presence of the analyte, in which prior to the application to the device of a liquid sample suspected of containing the analyte, the labeled specific binding reagent is retained in the dry state in a macroporous body, e.g. of plastics material having a pore size of 10 microns or greater, through which the applied liquid sample must pass en route to the porous carrier material, the labeled specific binding reagent being freely soluble or dispersible in any liquid sample which enters the macroporous body.

As stated above, known techniques for detecting Bence-Jones proteins are technically difficult, slow, and expensive and with sensitivity below that specified as clinically significant (typical gel electrophoresis sensitivity is 30-50 mg/L, it is recognized that 10 mg/L BJP is significant). Since the presence of Bence Jones protein in the urine may not be supported or suggested symptomatically, a large percentage of positive samples remain unidentified in the early stages. The present invention relates generally to assay devices and specifically to those devices making use of chromatographic techniques in conducting specific binding assays for the determination of the presence of free light chains in untreated urine. In particular, this technique provides kits for rapid determination between free light chain, and between classes of light chain. It has been surprisingly found that the use of chromatographic assays for the detection of Bence Jones proteins provides improved rapid assays that require little technical expertise, reduced risk of contamination, improved sensitivity, and may be used either as a screen, or as a confirmatory step prior to immunoelectrophoresis, or immunofixation electrophoresis utilizing untreated human urine.

### SUMMARY OF THE INVENTION

It is the object of the present invention to provide analytical test device incorporating a dry porous carrier to which a liquid sample suspected of containing Bence Jones proteins can be applied indirectly, the device also incorporating a labeled specific binding reagent in a conjugate pad which is freely mobile in the porous carrier when in the moist state, and an unlabelled specific binding reagent which is permanently immobilized in a detection zone on the dry porous carrier material. The labeled and unlabelled specific binding reagents being capable of participating in a competition reaction in the presence of the analyte, in which prior to the application to the device of a liquid sample suspected of containing the analyte, the labeled specific binding reagent is retained in the dry state in a conjugate pad through which the applied liquid sample must pass en route to the porous carrier material, the labeled specific binding reagent being freely soluble or dispersible in any liquid sample which enters the conjugate pad.

It is the object of the present invention to provide a test strip for the detection of Bence Jones proteins and classes thereof (lambda/kappa) having at least one conjugate pad containing in the dry state a labeled specific binding reagent that is freely soluble or dispersible in an aqueous sample that may be applied to the conjugate pad. The invention further encompasses any analytical device that incorporates such a conjugate pad together with a test strip or the like into which liquid sample carrying dissolved or dispersed labeled specific binding reagent can flow from the conjugate pad. The invention also encompasses the use of such a conjugate pad to facilitate the uptake of a labeled specific binding agent by a liquid sample before such a sample is analyzed on a test strip or the like for the presence of Bence Jones proteins.

It is the object of the present invention to provide multiple test strips run side-by-side having conjugate pads in the dry state containing various labeled specific binding reagent specific to Bence Jones proteins which are freely soluble in liquid sample that may be applied to the pads. The invention further encompasses any analytical device that incorporates running two test strips side-by-side in order to determine class of free light chain in an untreated urine sample. The invention encompasses any analytical test device which indicates the presence of both kappa and lambda monoclonal free light chains.

It is the object of the present invention to provide a device for the detection of Bence Jones proteins in a urine sample comprising: a first conjugate pad comprising a first labeled binding reagent capable of binding to a first analyte; a second conjugate pad comprising a second labeled binding reagent capable of binding to a second analyte; a first dry porous strip comprising a matrix through which sample can pass by capillarity capable of carrying the first labeled binding reagent and the first analyte, wherein the dry porous strip comprises two reaction sites, a first reaction site comprising a first immobilized binding reagent capable of immobilizing the first labeled binding reagent in relation to the absence of the first analyte in the sample, a second reaction site comprising an nonspecific immobilized binding reagent capable of immobilizing immunochemicals; a second dry porous strip comprising a matrix through which sample can pass by capillarity capable of carrying said second labeled binding reagent and said second analyte, wherein said second dry porous strip comprises two reaction sites, a third reaction site comprising a second immobilized binding reagent capable of immobilizing the second labeled binding reagent in relation to the absence of the second analyte in the sample; a fourth reaction site comprising a nonspecific immobilized binding reagent capable of immobilizing immunochemicals.

It is the object of the present invention to provide a kit for determining the presence of Bence Jones protein in urine comprising; at least one analytical test strip suitable for analyzing a liquid sample suspected of containing a Bence Jones protein, the test strip comprising the following separate components: (1) a liquid sample receiving member; (2) a conjugate pad positioned to receive liquid sample from the liquid sample receiving member, the liquid receiving member including a mobile labeled specific binding reagent for binding to analyte in the sample; and (3) a first dry porous carrier strip downstream of the conjugate pad, the carrier strip including a first detection zone comprising an unlabeled immobilized specific binding reagent for binding to the first mobile labeled specific binding reagent in relation to the absence of analyte in the liquid sample, the first mobilizable labeled reagent being freely soluble or dispersible in liquid sample applied to the liquid sample receiving member and free to move therewith through the pores of the first conjugate pad whereby it is transported by the liquid sample from the first conjugate pad to the detection zone, the conjugate pad and carrier strip comprising separate and different materials which overlap at their adjacent ends to provide effective contact between these components to ensure that liquid sample applied to the liquid sample receiving member can permeate sequentially through the conjugate pad and the first carrier strip; (4) an adsorbent pad disposed upon the dry porous strip such that the absorbent pad is positioned opposite the conjugate pad and such that the reaction site lies in-between the conjugate pad and the first absorbent pad; and a housing suitable for holding at least one test strip and applying pressure to the test strip components, the housing comprising the following separate parts: (1) a substrate; (2) at least one well within the substrate capable of encasing a test strip; (3) a cap for covering the substrate, the cap capable of exerting pressure upon the test strip components to ensure an optimal lateral flow of liquid sample throughout test strip components.

While the following terms are believed to be well understood by one of skill in the art, the following definitions are set forth to facilitate explanation of the invention.

The term “Bence Jones protein” means small protein, composed of a light chain of immunoglobulin, made by plasma cells.

The term “Multiple myeloma” means a tumor of the plasma cells.

The term “flowability” means the ability to flow.

### BRIEF DESCRIPTION OF THE DRAWINGS

FIG. 1 is a front plan view of one form of the test strip device of the present invention.

FIG. 2 is a cross-sectional view of one form of the test strip device shown in FIG. 1.

FIG. 3 is a front plan view of one form of the test strip device of the present invention.

FIG. 4 is a front plan view of one form of the test strip device of the present invention showing the front face of the invention.

FIG. 5 is an exploded view of one form of the test strip device of the present invention showing the front face of the invention.

### DETAILED DESCRIPTION

It is well known that Bence Jones proteins are small proteins (light chains of immunoglobulin) found in the urine. Testing for these proteins is done to diagnose and monitor multiple myeloma and other similar diseases. Bence Jones proteins are considered the first tumor marker. Bence Jones proteins are made by plasma cells, a type of white blood cell. The presence of these proteins in a person's urine is associated with a malignancy of plasma cells. Multiple myeloma, a tumor of plasma cells, is the disease most often linked with Bence Jones proteins. The amount of Bence Jones proteins in the urine indicates how much tumor is present. Physicians use Bence Jones proteins testing to diagnose the disease as well as to check how well the disease is responding to treatment.

It is well known that urine is the best specimen in which to look for Bence Jones proteins. Proteins are usually too large to move through the glomerular filter in the kidney, from the blood into the urine. Bence Jones proteins are an exception. They are small enough to move quickly and easily through the kidney into the urine.

Immunoglobulin molecules normally consist of pairs of polypeptide chains of unequal size bound together by several disulphide bridges. In each immunoglobulin molecule there are a pair of heavy chains which may be either gamma, alpha, mu, delta or epsilon type, and a pair of light chains which may be either kappa or lambda. In some pathological conditions such as Multiple Myeloma, there is a proliferation of one antibody-producing plasma cell leading to excess production of light chains of one specific kind. These free monoclonal light chains can be found in urine and plasma and are known as Bence-Jones Proteins (BJP). The present invention provides improved chromatographic assays for the detection of Bence Jones proteins in urine.

Preferably, test strips according to the present invention are utilized for the detection of an analyte of interest in a sample. The analyte of interest is preferably free light chains (kappa and/or lambda), however one preferred embodiment of the present invention provides a control zone capable of detecting or binding to a large class of immunochemicals, including proteins and antibodies. The detection of the presence of an analyte of interest in the detection zone may indicate the presence or absence of disease. The detection of the presence of an analyte in the control zone may also indicate that the test strip is working.

Preferably, chromatographic test strips of the present invention are used to detect Bence Jones proteins in untreated urine. The test strips of the present invention utilize a dry porous carrier material, such as a strip of nitrocellulose. If desired, the nitrocellulose can be backed with moisture impermeable material, such as polyester sheet. This backing is preferably transparent to facilitate a densitometric analysis, or transmission of light for quantitation of total Bence Jones protein within the porous carrier detection zone. Using nitrocellulose as the porous carrier material has considerable advantage over more conventional strip materials, such as paper, because nitrocellulose has a natural ability to bind proteins without requiring prior sensitization. Specific binding reagents, such as immunoglobulins, can be applied directly to nitrocellulose and immobilized thereon. No chemical treatment is required which might interfere with the essential specific binding activity of the reagent. Moreover, nitrocellulose is readily available in a range of pore sizes, which facilitates the selection of a carrier material to suit particularly requirements such as sample flow rate. Preferably the nitrocellulose has a pore size of at least one micron. Preferably the nitrocellulose has a pore size not greater than about 20 microns.

In a preferred embodiment of the invention, the labeled specific binding reagent comprises a specific binding reagent attached to a particulate label. Such “direct labels”, e.g. gold sols, and non-metallic colloids, are already known per se. They can be used to produce an instant analytical result without the need to add further reagents in order to develop a detectable signal. They are robust and stable and can therefore be used readily in an analytical device which is stored in the dry state. Their release on contact with an aqueous sample can be modulated, for example by the use of soluble glazes. Preferably, the particulate label is a gold sol which can be readily visible to the eye if it becomes bound in the detection zone. If desired, the assay result can be read instrumentally, e.g. by color reflectance, or scanned into a computer to measure band intensity. The reaction zone and control zone may be viewed manually or by an optical/electronic reader. The purpose of a reader is to allow consistency and quantification of the reading. Readers in accordance with the present invention include scanner, image analysis, or a camera, such as a digital camera. A less expensive way to read the test results is to use a document scanner such as an HP scanner. This type of scanner connects to a PC via the printer port or the USB port. The assays of the present invention may be placed upside down on the reader and scanned. The result is an image file that can be viewed on the PC and analyzed by image analysis software. Another reader of the present invention runs an application program (run on a PC) that would control the image capture and presentation of data to the user. In an application program, results can be stored in a PC and used to evaluate the progress of the disease and/or treatment if the test is repeated periodically. A third reader would be a digital camera. The camera would be positioned over the test and control areas of the present invention. Digital images can be stored and retrieved at a later date to monitor disease or treatment thereof.

In a preferred embodiment of the invention, a chromogenic mobile specific binding partner, or mobilizable labeled specific binding reagent is applied to a conjugate pad. Conjugate pads according to the present invention may be made from materials, which include glass fibers, paper, or polypropylene filters. Preferably conjugate pad is manufactured from glass fibers. Conjugate pad is presoaked in solution containing at least one mobile specific binding partner, which is specifically reactive with analytes of interest, for example lambda, or kappa monoclonal free light chains.

When the labeled specific binding reagent is impregnated and dried into conjugate pad, the test strips are optimized for competitive binding type assays. According to such methods, an immobilized specific binding reagent is selected to competitively bind with labeled binding reagent and analyte of interest. Likewise, the labeled specific binding reagent is preselected to be a specific binding analogue of the analyte that will bind competitively with the immobilized specific binding reagent located in the porous membrane matrix. The mobile specific binding partner is preselected from anti-free kappa and/or anti-free lambda antibodies conjugated with a visible colloidal metal. Although other colloidal metals that one of skill in the art would use to bind to an antibody to make a visible color may work, gold is preferred with the present invention for it provides an easily discernable red band in relation to the presence of analyte. In one preferred embodiment the mobile specific binding partner may be one conjugated polyclonal antibody. In another preferred embodiment, the mobile specific binding partner may be one conjugated monoclonal antibody or a conjugated monoclonal antibody cocktail.

By incorporating a mobile specific labeled reagent in a separate conjugate pad, rather than pre-dosed onto the carrier material that also incorporates the detection zone, the following advantages can be obtained: (1) ease of manufacture, because Bence-Jones Protein assays require the analysis of different classes of free light chains, i.e. both the kappa and the lambda type, the incorporation of the labeled reagent specific to each in separate conjugate pads avoids the need to apply the labeled reagent in a special zone in the carrier, which may need careful pre-treatment; (2) uniformity in test strip assembly, because the remaining components in the test strip have similar dimensions, they can be easily manufactured and assembled into test strips; and (3) enhancing sensitivity of the test because a substantial quantity of the liquid sample is able to take up the chromogenic mobile labeled specific binding reagent before migrating through the carrier material to the detection zone, enhancing potential reaction time without significantly increasing overall test time.

In the preferred embodiment, the assay device identifies one analyte per test strip. This is done by having one labeled binding reagent in one conjugate pad, and one immobilized specific binding reagent in the porous carrier. Multiple test strips may be run side by side which are manufactured to identify the presence of various analytes of interest. Preferably, a kappa sensing test strip is run next to a lambda sensing test strip. Side by side analysis allows for a reduced risk of contamination and is easier to use than state of the art test kits.

In the preferred embodiment of the present invention, the assay device identifies more than one analyte (e.g. kappa and lambda monoclonal free light chains) in a single sample by a competitive binding type assay. This is done utilizing a device capable of running two or more test strips simultaneously, in a side-by-side fashion. Test strip one contains a first conjugate pad having a first labeled specific binding reagent impregnated and dried into the conjugate pad. Test strip one also has a first immobilized specific binding reagent in the dry porous carrier preselected to specifically bind with the first labeled specific binding reagent. The first labeled specific binding reagent, however, is preselected to specifically bind with analyte that will bind competitively with the first immobilized specific binding factor. Preferably first labeled specific binding factor will bind to monoclonal free lambda light chains. Accordingly when a sample containing monoclonal free lambda light chains is added to test strip one, the lambda free light chains in the sample will bind with the first labeled specific binding reagent upon contact and form an analyte/labeled binding reagent complex. This complex formation decreasing the ability of first labeled specific binding reagent to bind with first immobilized specific binding partner. Although not wishing to be bound by any theory, it is believed that in a positive sample containing monoclonal free lambda light chains, the epitopes or binding areas on the first labeled specific binding reagent become saturated with analyte, and lose the ability to also bind with first immobilized specific binding reagent upon contact. Accordingly, in a negative sample, first labeled specific binding reagent does not bind to analyte in sample, and is free to bind with the first immobilized specific binding reagent upon contact therewith. Test strip two contains a second conjugate pad having a second labeled specific binding reagent impregnated and dried into the second conjugate pad. Test strip two also has a second immobilized specific binding reagent in the dry porous carrier selected to specifically bind with the analyte of interest such as free kappa light chains. The second labeled specific binding reagent, however, is preselected to specifically bind with analyte that will bind competitively with the second immobilized specific binding factor. Preferably this second labeled specific binding factor will bind to monoclonal free kappa light chains. Accordingly when a sample containing monoclonal free kappa light chains is added to test strip two, the kappa free light chains in sample will bind with the second labeled specific binding reagent upon contact to form a analyte/labeled binding reagent complex. Complex formation decreasing the ability of second labeled specific binding reagent to bind with second immobilized specific binding reagent. Although not wishing to be bound by any theory, it is believed that in a positive sample containing monoclonal free kappa light chains, the epitopes or binding areas on the second labeled specific binding reagent become saturated with analyte, and lose the ability to also bind with second immobilized specific binding reagent upon contact. Accordingly, in a negative sample, second labeled specific binding reagent does not bind to analyte in sample, and is free to bind with the second immobilized specific binding reagent upon contact therewith.

In alternative embodiments of the present invention, conjugate pad can incorporate several labeled mobile specific binding reagents to indicate the presence of multiple analytes in a single sample. For example, both kappa and lambda monoclonal free light chains can be detected. This will facilitate the manufacture of a multiple analyte testing devices for analytes of interest, e.g. kappa and lambda detection in a single test strip. In this embodiment, immobilized specific binding reagents are striped at various locations along the porous carrier to create multiple detection zones. For example, it a first immobilized specific binding reagent may be striped along the porous carrier at a position closer to the conjugate pad than a second immobilized specific binding reagent. In this case, the first immobilized specific binding reagent is said to be upstream from the second immobilized specific binding reagent. Conversely, the second immobilized specific binding reagent is said to be downstream of the first immobilized specific binding reagent.

Ideally, conjugate pad of the present invention is in direct moisture-conductive contact with the porous material, and the detection zone on the porous carrier material is spaced away from the region of contact between the porous carrier material and the conjugate pad. In such an embodiment, the quantity of liquid sample required to saturate the conjugate pad is preferably not less than the quantity of liquid sample capable of being absorbed by the mass of porous carrier material linking the conjugate pad and the detection zone. In other words, the liquid capacity of the conjugate pad is at least equal to the liquid capacity of the working portion of the porous carrier. The conjugate pad is optimized to hold approximately between 100 to 300 μl of liquid sample, preferably about 200 μl.

In one preferred embodiment of the present invention the test strip may further comprise an absorbent pad. Absorbent pad is μlaced at the far end of the immunochromatographic strip. The major advantage of using an absorbent pad is that the total volume of sample that enters the test can be increased. This increased volume can be used to wash away unbound detector reagent from the nitrocellulose membrane. The net result is that the assay readout zone will have a lower background and assay sensitivity can be enhanced. Since the volume of sample that ultimately contributes to signal is controlled by the volume required to solubilize the detector reagent, and not by the total volume of sample that enters the device, the addition of the absorbent pad may not have a dramatic impact on overall assay sensitivity.

In the preferred embodiment of the invention, the labeled reagent is a specific binding partner for the analyte. The labeled reagent, the analyte (if present) and the immobilized unlabelled specific binding reagent compete together in a “competition” reaction. This results in the labeled reagent being bound in the detection zone if analyte is not present in the sample. The labeled specific binding partner is selected to be a specific binding analogue of the analyte that will bind competitively with the immobilized specific binding reagent located in the porous membrane matrix.

In a further embodiment of the present invention, the porous carrier is linked via the conjugate pad to a porous receiving member to which the liquid sample can be applied and from which the sample can permeate into the porous carrier. Preferably, the porous carrier and the conjugate pad are contained within a moisture-impermeable casing or housing and the porous receiving member can be μlaced below a gap in the housing or front plate and can act as a means for permitting a liquid sample to enter the housing and reach the porous carrier. Sample receiving member can be made from any porous or fibrous material capable of absorbing liquid rapidly. The porosity of the material can be unidirectional (i.e. with pores running wholly or predominantly parallel to an axis of the member) or multidirectional (omnidirectional, so that the member has an amorphous sponge-like structure). Porous sample receiving members can be made from paper or other cellulosic materials, such as nitrocellulose. Preferably the material comprising the porous receiving member should be chosen such that the porous member can be saturated with aqueous liquid within a matter of seconds. The liquid must thereafter permeate freely from the porous sample receiving member into the conjugate pad. Typically the porous receiving member is located under the gap in the housing where sample is first added to the test strip or system. The porous receiving member is the first area of the test strip to contact liquid sample. The porous receiving member is in contact with the conjugate pad such that sample can flow between the two pads. The conjugate pad is disposed upon the porous dry carrier so that liquid can flow between that conjugate pad and the porous dry carrier.

In the preferred embodiment, the housing should be provided with means, e.g. appropriately placed aperture or apertures, which enable the detection zone of the porous solid phase carrier material (carrying the immobilized unlabelled specific binding reagent) to be observable from outside the housing so that the result of the assay can be observed. The housing being constructed of opaque or translucent material incorporating at least one aperture through which the analytical result may be observed, together with at least one gap above the receiving member. The housing may be constructed to have a transparent back late to facilitate a densitometric analysis, or transmission of light through the test strip to facilitate quantitation of total Bence Jones proteins. If desired, the housing may also be provided with further means which enable a further zone of the porous solid phase carrier material to be observed from outside the housing and which further zone incorporates one or more control reagents which enable an indication to be given as to whether the assay procedure has been completed. Preferably the housing provides at least one well capable of holding at least one test strip of the present invention. The housing may have at least two wells capable of holding a test strip each, so that two test strips can be run side-by-side within the same housing.

In the preferred embodiment, the housing performs a critical task of ensuring contact among the system components. The tests strips of the preferred embodiment typically comprise a receiving member, a conjugate pad, a porous carrier and an absorbent pad. It is important that these components be in sequential fluid communication. This means that when liquid sample is applied to the system, liquid is able to travel by capillarity between various system components because they are in contact. The housing μlaces pressure in the system where the various test strip components overlap. If the pressure is too high, the components can be crushed, which can block pores and the ability of liquid sample to flow. A crushed conjugate pad may prevent contact between positive sample and system components such as porous carrier and absorbent pad. This may contribute to false positive results. A crushed porous member may also prevent the movement of analyte or labeled binding reagent throughout the porous member further causing inaccurate results. Conversely, if the pressure in the system is too low, the system may lose sequential fluid communication. In the preferred embodiment, the housing provides optimum pressure to ensure flowability of liquid sample throughout the overlapping components in the test strip. It is understood in the art, that flowability of liquid sample, analyte, and complexes throughout system components can be adjusted by trial and error. One of skill in the art may adjust flowability by altering porosity of each component, size of each component, dimensions of each component, including thickness of each component, as well as pressure on system components.

In the preferred embodiment the housing comprises a hollow elongated casing containing at least two wells. Each well is capable of supporting a test strip having a dry porous nitrocellulose carrier which communicates indirectly with the exterior of the casing via a gap and a receiving member. The porous nitrocellulose carrier and the sample receiving member being linked via a conjugate pad such that any sample reaching the porous carrier must first pass through the conjugate pad, and the sample receiving member. The conjugate pad and sample receiving member when taken together act as a reservoir from which urine is released into the porous carrier. The conjugate pad containing a highly-specific anti-Bence Jones antibody bearing a colored “direct” label, the labeled antibody being freely mobile within the conjugate pad and the porous carrier when in the moist state. The porous carrier containing a detection zone on the carrier spatially distant from the conjugate pad and a highly-specific unlabelled monoclonal free light chain protein which is permanently immobilized on the carrier material and is therefore not mobile in the moist state. The monoclonal free light chain protein is preselected from either monoclonal free lambda, or kappa light chains.

In the preferred embodiment, the porous dry carrier further comprises a control zone designed to convey an unrelated signal to the user that the device has worked. For example, the control zone can be loaded with protein that will bind to the labeled reagent, e.g. Protein A, to confirm that the sample has permeated the test strip. As the purpose of the control zone is to indicate to the user that the test has been completed, the control zone should be located downstream from the detection zone in which the desired test result is recorded. A positive control indicator therefore tells the user that the sample has permeated the required distance through the test device.

In the preferred embodiment, labeled specific binding reagent comprises a chromogenic label. The label can be any entity the presence of which can be readily detected. Preferably the label is a direct label, ie. an entity which, in its natural state, is readily visible either to the naked eye, or with the aid of an optical filter, reader, and/or applied stimulation, e.g. UV light to promote fluorescence. For example, minute colored particles, such as metallic sols (e.g. gold), are very suitable. Concentration of the label into a small zone or volume should give rise to a readily detectable signal, e.g. a strongly colored area. This can be evaluated by eye, or by instruments if desired. In the competitive reaction, a signal indicates the absence of analyte, because the labeled binding reagent has not bound to the analyte of interest and is free to bind to the immobilized specific binding reagent located in the reaction zone. Conversely, the lower the signal, the more analyte in the sample, because the labeled binding reagent has bound to the analyte of interest and cannot also bind to the immobilized specific binding reagent located in the reaction zone. Accordingly, the presence or intensity of the signal from the label which becomes bound in the detection zone can provide a qualitative or quantitative measurement of analyte in the sample.

In all embodiments of the invention, it is essential that the labeled reagent and analyte complexes thereof, and analyte (if any), migrate with the liquid sample as it progresses to the detection zone. Preferably, the flow of sample continues beyond the detection zone and sufficient sample is applied to the porous carrier material in order that this may occur and that any excess labeled reagent which does not participate in any binding reaction in the detection zone is flushed away from the detection zone by this continuing flow. Preferably an absorbent pad can be provided at the distal end of the carrier material. The absorbent sink may comprise, for example, Whatman 3 MM chromatography paper, and should provide sufficient absorptive capacity to allow any unbound conjugate to wash out of the detection zone.

The immobilized reagent in the detection zone is preferably a free light chain protein. Preferably, a free light chain protein capable of binding competitively to labeled binding reagent when similar monoclonal free light chain protein is present in a sample. The monoclonal free light chain is extracted and purified using techniques known in the art of extracting and purifying proteins. The free light chain is typically striped along the narrow width of the porous carrier material. Preferably, the free light chain is stripped along the dry porous carrier to create a detection zone approximately 1.0 mm in width. When a solution of free light chain is striped along dry porous carrier it becomes permanently immobilized therein. Preferably the porous carrier material is in the form of a strip or sheet to which during manufacture of the device, one or more reagents can be similarly applied in distinct zones. During use, the liquid sample is allowed to permeate through the sheet or strip from one side or end to another.

In the preferred embodiment multiple test strips are run side by side, having separate conjugate pads impregnated with at least one different chromogenic mobile specific binding reagents for binding to multiple analytes of interest such as kappa and lambda monoclonal free light chains. Preferably in the present invention, there are two test strips μlaced side by side in the housing. The first test strip has a first conjugate pad, containing a first mobile specific binding reagent. The first mobile specific binding reagent is chromogenic and capable of binding to lambda light chains. The second test strip comprises a second conjugate pad comprising a second chromogenic mobile specific binding reagent. The second mobile specific binding reagent is chromogenic and capable of binding to kappa light chains.

When multiple test strips are run side by side, multiple reaction sites may be included to detect multiple analytes of interest. Preferably in the present invention, there are two test strips laced side by side in the housing where each strip contains two reaction zones approximately 1.0 mm in width. The first test strip has a first reaction zone or site and a second reaction zone or site. The first reaction site comprises a first immobilized specific binding reagent capable of immobilizing the labeled binding reagent from the conjugate pad in relation to the absence of analyte in the sample. The second reaction zone is preferably a control zone designed merely to convey an unrelated signal to the user that the device has worked. For example, the control zone can be loaded with an protein that will bind to the mobile labeled binding reagent, e.g. Protein A, to confirm that the sample has permeated the test strip.

The second test strip has a third reaction zone and a fourth reaction site. The third reaction site comprises a second immobilized specific binding reagent capable of immobilizing the mobile specific binding reagent from the second conjugate pad in relation to the absence of analyte in the sample. The fourth reaction zone is preferably a control zone designed merely to convey an unrelated signal to the user that the device has worked. For example, the control zone can be loaded with an protein that will bind to the labeled reagent, e.g. Protein A, to confirm that the sample has permeated the test strip. The designation of “first”, “second”, “third” and “fourth” reaction zone is not meant to limit the present invention in any way. The designation is meant to show that there are multiple tests strips of substantially similar design operating in unison, each test strip preferably having a reaction zone and a control site. For example, if three test strips were run simultaneously, the reaction zones would be designated and the fifth and sixth reaction zones because two zones are added with the addition of an additional strip.

In the preferred embodiment, porous dry carrier has a first end at which chromatographic urine transport begins and a second end at which chromatographic solvent transport ends. The length of porous dry carrier comprises a first reaction site, and a second reaction site. Second reaction site acts as a control that indicates that sample has been transported throughout the length of test strip. All parameters on the porous dry carrier or cellulosic membrane may be changed but the width will not affect the sensitivity (provided it is above ˜4 mm, where edge drag effects come in to play). Absorbent pad is placed upon cellulosic membrane opposite conjugate pad such that capillary action can draw sample from first end to second end thereby transporting sample through reaction site, and second reaction site. Absorbent pad can be fabricated from any material that has a propensity to wick liquid such as a sponge or paper towel material and is typically 1 7mm. long and 6 mm. wide. The size and thickness must be such that it provides enough capillarity to transport sample from first end to second end.

The first reaction site is impregnated with a first immobilized specific binding reagent capable of reacting with and the immobilization of the mobile labeled binding reagent when analyte such as free light chain is absent in sample. In test strips of the present invention the immobilized specific binding reagent is free kappa or free lambda light chain proteins. In the presence of free light chains in a sample, the immobilized free light chains bindi competitively with the labeled binding reagent, and to the free kappa or lambda light chain proteins in the sample The second reaction site, or control site is downstream of the first reaction site and is impregnated with a second immobilizing specific binding factor. Typically a chemical capable of specifically binding to immunochemicals, such as Protein A. Although not wishing to be bound by any theory, Protein A is capable of effectively collecting and immobilizing antigen-antibody complexes by binding to the Fc section of immunoglobulins. Control reaction site acts as a positive control and is relied upon to indicate that capillary action has carried test sample, mobile specific binding partners, and analyte/chromogenic mobile specific binding partner complexes thereof throughout the length of test strip.

The invention also provides an analytical method in which a device as set forth above is contacted with an aqueous liquid sample suspected of containing the analyte, such that the sample permeates by capillary action via the conjugate pad through the porous solid carrier into the detection zone and the labeled reagent migrates therewith to the detection zone, the presence of analyte in the sample being determined by observing the extent (if any) to which the labeled reagent passes through the detection zone, and does not bind thereto. Accordingly, the absence of analyte in the sample being determined by observing the extent (if any) to which the labeled reagent becomes bound in the detection zone. In such methods, liquid sample suspected of containing analyte is first obtained. Preferably the liquid sample is urine because Bence Jones proteins are often small enough to pass through the glomerular filter and pass with urine from the body. Sample is obtained by collecting urine midstream. Next, aliquots of urine are portioned into between 100 to 300 microliters aliquots using a syringe or pipette, preferably 200 microliters. Next liquid sample is injected into the device using the syringe or pipette. Preferably 200 microliters of sample is injected upon the first receiving member located below the first gap. In order to apply sample to the second receiving member, the aliquoting steps are repeated and sample is applied to second receiving member. It is not necessary to change the syringe or pipette between applications to the receiving members because the same sample is applied to the separate receiving members.

After applying sample to the device, sample permeates laterally throughout the device by capillary action via the conjugate pad through the porous solid carrier into the detection zone and the labeled reagent migrates therewith to the detection zone, the presence of analyte in the sample being determined by observing the extent (if any) to which the labeled reagent passes through the detection zone, and does not bind thereto. This is because in a positive sample, analyte of interest will bind to labeled binding reagent to form an analyte/labeled binding reagent complex. Such complexes have a decreased ability to bind to the immobilized reagent in the porous carrier. It has been surprising found that a lateral flow device of the present invention provides quick, and accurate indication for the presence of free light chains, and classes of free light chains.

By way of example only, some preferred embodiments of the invention will now be described in detail with reference to the accompanying drawings.

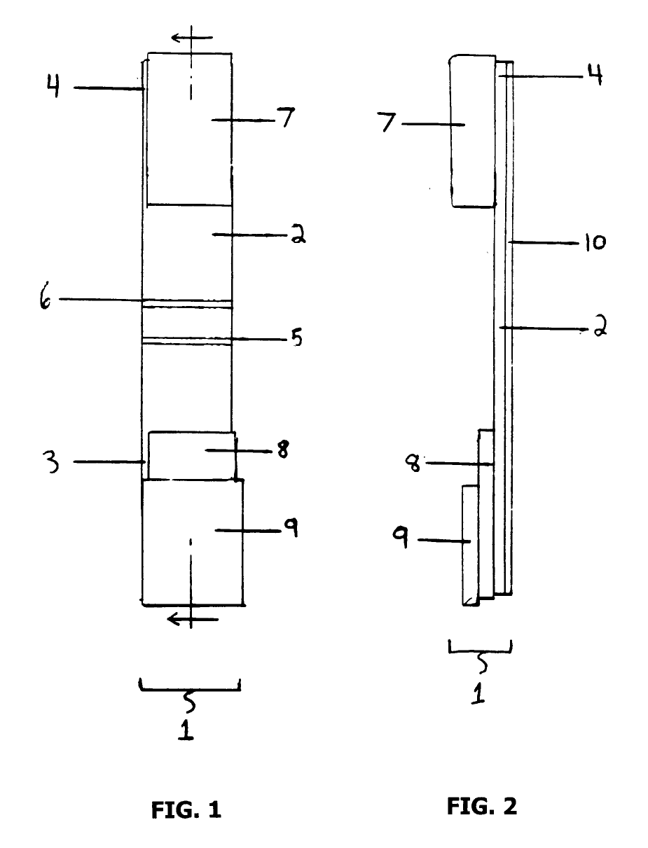

Referring to FIG. 1, the device comprises a test strip (1). Test strip (1) is preferably 60 mm. in length, and 6 mm. in width. Test strip (1) is largely comprised of a dry porous carrier (2) having a first end (3) at which chromatographic transport begins and a second end (4) at which chromatographic solvent transport ends. The length of dry porous carrier (2) comprises a first reaction site, or detection zone (5), and a second reaction site or control zone (6). Second reaction site (6) acts as a control that indicates that sample has been transported throughout the length of test strip (1). Typically dry porous carrier (2) is 55 mm. in length, and 6 mm. wide. All parameters on dry porous carrier (2) may be changed but the width will not affect the sensitivity (provided it is above ˜4 mm, where edge drag effects come in to play). Absorbent pad (7) is placed upon dry porous carrier (2) opposite conjugate pad (8) such that capillary action can draw sample from first end (3) to second end (4) thereby transporting sample through first reaction site (5), and second reaction site (6). Absorbent pad (7) can be fabricated from any material that has a propensity to wick liquid such as a sponge or paper towel. Located on the opposite end of test strip (1) it can be seen that receiving member (9) is disposed on top of conjugate pad (8). Receiving member (9) is preferably 10 mm. long and 5 mm. in width. Receiving member (9) is the first contact point for sample added to test strip (1).

Referring to FIG. 2, it can be seen that the dry porous carrier (2) of test strip (1) can have a backing (10) equal in length and width to dry porous carrier (2). Backing (10) may be added to add tensile strength to porous carrier (2) and may be made out of any plastic material. It can also be seen that absorbent pad (7) is placed at second end (4) upon dry porous carrier (2) opposite conjugate pad (8). Absorbent pad (7) may be longer than conjugate pad (8), in the range of 4 to 15 mm. preferably 10 mm. in length. Preferably absorbent pad (7) is about 5 mm. wide. Absorbent pad (7) may be thicker than conjugate pad (8). It can also be seen that receiving member (9) is located closer to first end (3) and may over hang the end of conjugate pad (8). Receiving member (9) may be between 5 mm to 15 mm in length, preferably 10 mm. Receiving member (9) is preferably 5 mm. wide.

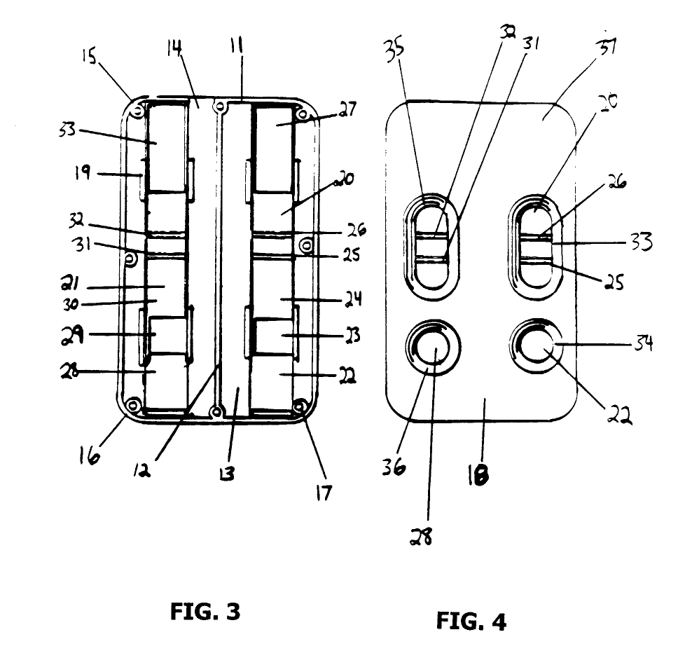

Referring to FIG. 3, it can be seen that one embodiment of the device comprises a housing, casing, or substrate (11) of elongate rectangular form is of hollow construction. Housing (11) may be between 30 mm. to 50 mm. wide, preferably about 37 mm wide. Housing (11) may be between 55 mm. to about 70 mm. in length, preferably about 65 mm. It can be seen that housing (11) also has at its center a divider (12). Substrate (11) can be made out of any material capable of retaining a liquid such as plastic or glass, preferably, substrate (11) is made out of plastic. Divider (12) is positioned on top of substrate (11 ) in order to form first well (13) and second well (14). Divider (12) is preferably positioned in the center of substrate (1 1 ) and runs from the top end (15) to the bottom end (16) of substrate (11). Divider (12) is preferably positioned so that well (13) is the same size as well (14). Preferably well (13) and well (14) are large enough so that each well can hold one test strip of the present invention. In the preferred embodiment of the present invention, substrate (11) comprises one divider (12), which makes two wells of equal size, where each well will hold one test strip (1). First well (13) is designed to hold first test strip (20). Second well (14) is designed to hold second test strip (21).

FIG. 3, also shows that at least one aperture (17) may be positioned around the edges of substrate (11). A cap (18) may be fitted onto substrate (11) and can abut against and fit into apertures (17). Furthermore, FIG. 3 also shows that at least one catch (19) positioned within each well for holding test strip (21) in place. Although numerous catches can be placed inside the wells, preferably, each test strip is given four catches (19) so that test strip (21) is held substantially in place. In this respect, the internal constructional details of housing (11) provides pressure to the test strip components which allows for adequate flowability of sample and analyte throughout the overlapping test strip components.

FIG. 3, also shows first test strip (20) contains a first receiving member (22) disposed upon a first conjugate pad (23), which is in turn is disposed upon a first dry porous carrier (24). Although not shown in FIG. 3. first dry porous carrier (24) comprising a matrix through which sample can pass by capillarity carrying a first mobile specific binding reagent and a first analyte, as well as complexes thereof. Dry porous carrier (24) comprises two reaction sites, a first reaction zone (25) located towards the middle of first test strip (20). First reaction site (25) comprises a first immobilized specific binding reagent capable of immobilizing a first chromogenic mobile specific binding partner when first analyte is absent in the sample. Dry porous carrier (24) further comprises a second reaction site (26) located downstream between the first reaction zone (25) and the first absorbent pad (27). Second reaction site (26) comprises an immobilized binding reagent capable of immobilizing immunochemicals. Located towards the top end (15) of substrate (11), and downstream on the first dry porous carrier (24) is a first absorbent pad (27). The first test strip (20) is designed similar to the test strip of FIGS. 1 & 2, therefore, sample applied to test strip (20) will first travel through the device in the same fashion. A negative sample will form a visible line on in first reaction zone (25). In a competition type reaction of the preferred invention, a visible line will form in the second reaction zone (26) as an indication that the sample has traveled through test strip (20).

FIG. 3, also shows second test strip (21) is similar in size and shape to first test strip (20). It can be seen that second test strip (21) contains a second receiving member (28) disposed upon a second conjugate pad (29), which is in turn disposed upon a second dry porous carrier (30). Second dry porous carrier (30) comprises a matrix through which sample can pass by capillarity carrying a second mobile specific binding partner and a second analyte. A third reaction zone (31) is located towards the middle of second test strip (21). Third reaction site (31) comprises a second immobilizing specific binding reagent capable of immobilizing a second mobile specific binding reagent in relation to the absence of analyte in a sample moving by capillary action, transporting the second chromogenic mobile specific binding reagent through the chromatographic test strip.

Located towards the top end (15) of substrate (11) is a second absorbent pad (33). A fourth reaction zone (32) is located downstream of the third reaction zone (31) between second absorbent pad (33) and third reaction zone (31). Fourth reaction site (32) comprises a binding reagent capable of immobilizing immunochemicals. Second test strip (21) is designed similar to the test strip of FIGS. 1 & 2 therefore, sample applied to test strip (21) will first travel through the device in the same fashion.

Although they are similar in size and construction, there are some important differences in the two strips, which are not shown in FIG. 3. First, conjugate pad (23) in test strip (20) is preferably manufactured to contain a first chromogenic mobile specific binding reagent capable of binding to a first analyte. Preferably the chromogenic mobile specific binding partner in conjugate pad (23) in test strip (20) is capable of specifically binding to lambda monoclonal light chains. Conversely, second conjugate pad (29) can be easily manufactured to contain a second chromogenic mobile specific binding reagent capable of binding to a second analyte. Preferably the chromogenic mobile specific binding reagent in second conjugate pad (29) is capable of specifically binding to kappa monoclonal light chain.

Referring to FIG. 3, the bottom end (16) and top end (15) of well (13) and well (14) respectively may be recessed to accommodate first end (3) and second end (4) within housing (11). Liquid sample applied to receiving members (22) can pass freely into conjugate pad (23) rapidly saturating conjugate pad (23). In turn, conjugate pads (23) is in liquid permeable contact with strip of first dry porous carrier (24). Upon saturation of first conjugate pad (23), liquid sample begins to permeate through the porous dry carrier (24) making it wet. Housing (11) is designed with divider (12) to ensure that first test strip (20) is not contaminated with liquid sample from second test strip (21) within housing (11). Divider (12) solves the problem of cross-contamination between test strips (20) and (21) in the same housing (11). Cross-contamination occurs when liquid sample saturates either test strip (20) or (21) and leaches from one test strip to the other with in housing (11). Leaching liquid may escape from the first test strip and carry along with it immunochemicals, and labeled specific binding reagent specific to that test strip, to the second test strip. Such cross contamination may provide false identification between classes of free light chains. Housing (11) is designed to ensure that liquid sample applied to receiving member (28) does not mix with sample applied to receiving member (22). FIG. 3, does not show backing (10) which would be positioned underneath first dry porous member (24) and second dry porous material (30). Since substrate (11) may provide adequate support for first dry porous member (24), and second dry porous material (30), backing (10) may not be included in all embodiments of the present invention.

Referring to FIG. 4, cap (18) is preferably fitted onto housing (11)(not shown). When cap (18) is fitted onto portion (11) of the housing, it covers first test strip (20), as well as second test strip (21), and becomes the upper face (37) of housing (11). Incorporated into upper face (37) is first window (33) and first gap (34). The first reagent containing detection zone (25), and second reaction zone (26) in first test strip (20), are positioned to be in window (33). When the device is used, liquid sample is added through gap (34). The sample immediately wets the first receiving member (22) of the first test strip (20). Although not visible in FIG. 4, a sample will travel through first receiving member (22), through first conjugate pad (23), through dry porous member (24) and travel by capillarity through the test strip (20) through the area visible in first window (33). A negative sample for lambda will show a visible banding pattern at the first reaction site (25). A visible banding pattern will form at second reaction zone (26) as an indication that sample has correctly passed through first test strip (20).

Incorporated into upper face (37) is second window (35) and second gap (36). The third reagent containing detection zone (31), and fourth reaction zone (32) in second test strip (21), are positioned to be in second window (35). When the device is used, liquid sample is added through gap (36). The sample immediately wets second receiving member (28) of the second test strip (21). Although not visible in FIG. 4, as sample will travel through second receiving member (28), through second conjugate pad (29), through second dry porous member (30) and travel by capillarity through the test strips through the area visible in second window (35). A negative sample for kappa will show a visible banding pattern at third reaction site (31). A visible banding pattern will form at fourth reaction zone (32) as an indication that sample has correctly passed through second test strip (21).

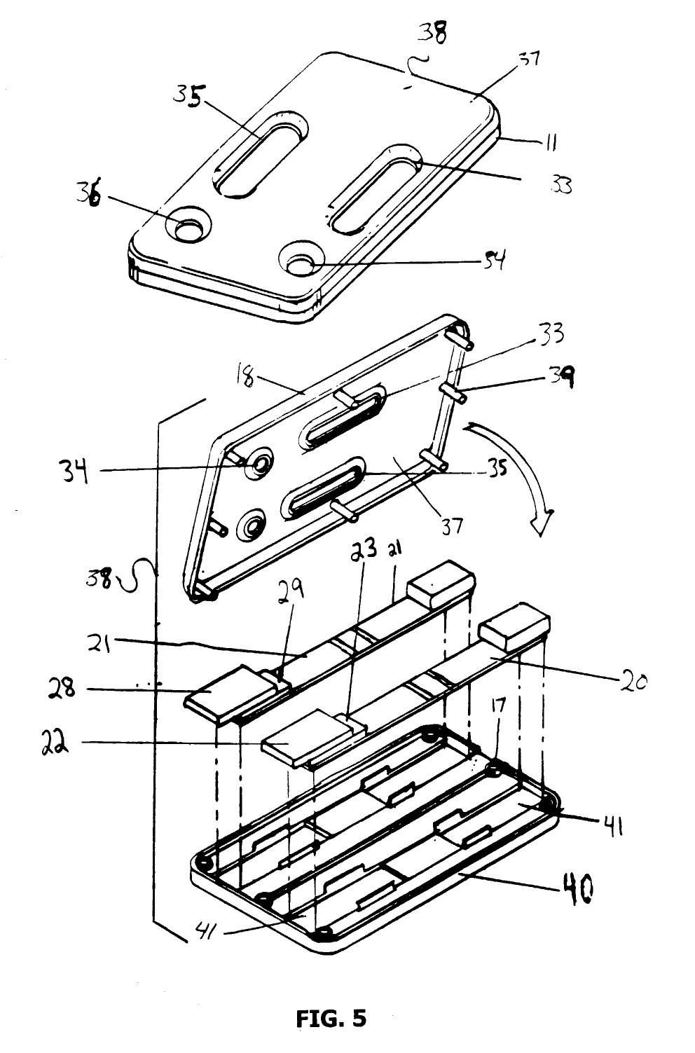

Referring to FIG. 5, the test device comprises a flat rectangular cassette (38), incorporating a first gap (34), a first window (33), a second gap (36) and a second window (35). The exploded view clearly shows at least one test strip of the present invention being positioned within cassette (38).

In operation, an aqueous sample can be applied through gap (34), e.g. by means of a syringe or pipette, to saturate first porous receiving member (22) which passes sample to first conjugate pad (23) containing labeled reagent which can be taken up by the sample. Thereafter, the aqueous sample can permeate the first test strip and, after an appropriate time, the test result can be observed through first window (33). Similarly, an aqueous sample can be applied through second gap (36), e.g. by means of a syringe, to saturate second porous receiving member (28) which passes sample to second conjugate pad (29) which contains labeled reagent which can be taken up by sample. Thereafter, the aqueous sample can permeate second test strip (21) and, after an appropriate time, the test result can be observed through second window (35). FIG. 5 further shows pin (39), which is designed to snap into aperture (17) to hold the top face (37) to the bottom plate (40). FIG. 5 also shows inner well (41), which optimizes pressure in component system, and ensures optimal flowability of sample throughout test strip components.

The following examples are given for the purpose of illustrating the present invention and are not intended to limit the scope in any way.

### EXAMPLE 1

Inoculated Goat Serum is Affinity purified against human BJP extract, suspended in a buffered saline solution and conjugated to 40 nm Gold colloid particles (Veda Labs, Alencon-France) by the following protocol. A ˜40 mn colloidal gold sol solution (externally Quality controlled by EM) is adjusted to pH 6 using 0.2M Sodium Hydroxide solution (using a Beckmann pH meter). The antibodies are introduced in the sol at circa 5-micrograms/ml concentration and incubated for 10 minutes. 10% Bovine serum albumin (Bayer fraction V) in tris buffered saline is then added to a final concentration of 0.2%. The solution is centrifuged for 30 minutes at 4° C. The supernatant is discarded and the pellet resuspended in BSA in Tris buffered saline. The suspension is then introduced into a glycerol gradient column and centrifuged for 45 minutes at 4° C. The purified band is then extracted from the gradient column.

### EXAMPLE 2

Basic Manufacturing Procedure: Inoculated Goat Serum is Affinity purified against human BJP extract, suspended in a buffered saline solution and conjugated to 40 nm Gold colloid particles. Conjugate pad material is liberally soaked in Conjugate solution and cut to about 7 mm length then affixed to CN membrane. Absorbent pads are attached to top and base of the device to allow sample introduction and osmotic conductance. Purified Human Bence-Jones Protein is suspended in Buffered saline and applied in a 1 mm line on the CN strip (test zone). This Antiserum is a Goat Polyclonal Anti-Human Free and Bound Light chain (But monoclonal or different host animal would be equally adequate). A 1 mm strip of Buffered saline suspended Protein A is applied at the control line. Strips are cut to a 6 mm width and placed in the cassette.

### EXAMPLE 3

Goat antibodies to the Free and Bound light chain classes were conjugated to 40 nm colloidal gold particles and introduced to a conjugate pad of glass paper. A 6 mm wide strip of cellulose nitrate was striped with individual lines of Protein A and urinary derived monoclonal light chain. An absorbent pad was attached to one end of the cellulose nitrate membrane. The glass pad was compressed against the cellulose nitrate membrane and urine seeded with monoclonal urinary derived light chains at concentrations 0 m/L, 10 mg/L, 20 mg/L, 30 mg/L, 50 mg/L, 100 m/L, 500 mg/L and 1000 mg/L. Urines were also seeded with albumin, IgA, IgG and IgM (polyclonal) at concentrations 0 mg/L, 10 mg/L, 20 mg/L, 30 mg/L, 50 mg/L, 100 m/L, 500 mg/L and 1000 mg/L. 300 microliters of seeded urine was introduced to the glass pad. Results are classified in table 1 below according to the absence of a clear line (positive) on the cellulose acetate where the competitive light chains were striped. Where the result was unclear an assignation ‘G’ is recorded. In all cases a line was observed at the Protein A line indicating that the conjugate/conjugate-antigen complex had correctly migrated through the test zone.

### EXAMPLE 4

Summary of Clinical results
Test sensitivity ˜10 mg/L Urinary free light chain

| Sensitivity | Specificity |
|---|---|
| 88%* | 100% |

Assay shows no cross reactivity between light chains, with pH, Sg, Albumin or any other physiological factors. *Samples demonstrated to have low levels of polyclonal (para) protein.

TABLE 1

| | Class | | | | | |
|---|---|---|---|---|---|---|
| mg/l | Kappa | Lambda | IgA | IgG | IgM | Albumin |
| 0 | − | − | − | − | − | − |
| 10 | + | − | − | − | − | − |
| 20 | + | G | − | − | − | − |
| 30 | + | + | G | − | G | − |
| 50 | + | + | + | G | + | − |
| 100 | + | + | + | + | + | − |
| 500 | + | + | + | + | + | − |
| 1000 | + | + | + | + | + | − |

What is claimed is:

1. A device for the detection of Bence Jones protein in a urine sample comprising:
a first conjugate pad comprising a first labeled binding reagent capable of binding to a first analyte;
a second conjugate pad comprising a second labeled binding reagent capable of binding to a second analyte;
a first dry porous strip comprising a matrix through which sample can pass by capillarity capable of carrying said first labeled binding reagent and said first analyte, wherein said dry porous strip comprises two reaction sites,
a first reaction site comprising a first immobilized binding reagent capable of immobilizing said first labeled binding reagent in relation to the absence of the first analyte in said sample,
a second reaction site comprising a nonspecific immobilized binding reagent capable of immobilizing immunochemicals;
a second dry porous strip comprising a matrix through which sample can pass by capillarity capable of carrying said second labeled binding reagent and said second analyte, wherein said second dry porous strip comprises two reaction sites,
a third reaction site comprising a second immobilized binding reagent capable of immobilizing said second labeled binding reagent in relation to the absence of said second analyte in said sample;
a fourth reaction site comprising a nonspecific immobilized binding reagent capable of immobilizing immunochemicals.

2. The device of claim 1 further comprising a first absorbent pad disposed upon said first dry porous strip such that said first absorbent pad is positioned opposite said first conjugate pad and such that said first reaction site, and said second reaction site lie in-between said first conjugate pad and said absorbent pad.

3. The device of claim 1 further comprising a second absorbent pad disposed upon said second dry porous strip such that said second absorbent pad is positioned opposite said second conjugate pad and such that said third reaction site, and said fourth reaction site lie in-between said second conjugate pad and said second absorbent pad.

4. The device of claim 1 further comprising a first receiving member disposed upon said first conjugate pad.

5. The device of claim 1 further comprising a second receiving member disposed upon said second conjugate pad.

6. The device of claim 1 further comprising a substrate.

7. The device of claim 6 wherein said substrate is selected from the group consisting of glass, wood, plastic, synthetic polymer, wood, natural and synthetic fibers, rubbers, and combinations thereof.

8. The device of claim 6 wherein the substrate further comprises at least two wells.

9. The device o f claim 6 wherein said substrate is made of transparent material.

10. The device of claim 1 further comprising a cap.

11. The device of claim 10, wherein said cap further comprises at least one window.

12. The device of claim 10, wherein said cap further comprises at least one gap.

13. The device of claim 1 wherein said sample is untreated human urine.

14. The device of claim 1 wherein said first labeled binding reagent is an anti-Bence Jones protein antibody.

15. The device of claim 14 wherein said anti-Bence Jones protein antibody is selected from the group consisting of monoclonal and polyclonal antibody.

16. The device of claim 14 wherein said anti-Bence Jones protein antibody is an anti-lambda Bence Jones antibody capable of binding to lambda free light chain.

17. The device of claim 1 wherein said second labeled binding reagent is an anti-Bence Jones protein antibody.

18. The device of claim 17 wherein said anti-Bence Jones protein antibody is selected from the group consisting of monoclonal and polyclonal antibody.

19. The device of claim 17 wherein said anti-Bence Jones protein antibody is an anti-kappa Bence Jones protein antibody capable of binding to kappa free light chain.

20. The device of claim 1 wherein said first immobilized binding reagent is lambda free light chain protein.

21. The device of claim 1 wherein said second immobilized binding reagent is kappa free light chain protein.

22. The device of claim 1 wherein said nonspecific immobilized binding reagent is Protein A.

23. The device of claim 1 wherein said first analyte is monoclonal lambda free light chain.

24. The device of claim 1 wherein said second analyte is monoclonal kappa free light chain.

25. The device of claim 1 wherein said first reaction site is capable of indicating the absence of said first analyte in said sample by the formation of a detectable band on said first dry porous carrier.

26. The device of claim 1 wherein said third reaction site is capable of indicating the absence of said second analyte in said sample by the formation of a detectable band on said second dry porous carrier.

27. A kit for determining the presence of Bence Jones protein in urine comprising;
at least one analytical test strip suitable for analyzing a liquid sample suspected of containing a Bence Jones protein, said test strip comprising the following separate components: (1) a liquid sample receiving member; (2) a conjugate pad positioned to receive liquid sample from said liquid sample receiving member, said liquid receiving member including a mobile labeled specific binding reagent for binding to analyte in said sample; and (3) a first dry porous carrier strip downstream of said conjugate pad, said carrier strip including a first detection zone comprising an unlabeled immobilized specific binding reagent for binding to said first mobile labeled specific binding reagent in relation to the absence of first analyte in said liquid sample, said first mobilizable labeled reagent being freely soluble or dispersible in liquid sample applied to said liquid sample receiving member and free to move therewith through the pores of said first conjugate pad whereby it is transported by said liquid sample from said first conjugate pad to said detection zone, said conjugate pad and carrier strip comprising separate and different materials which overlap at their adjacent ends to provide effective contact between these components to ensure that liquid sample applied to said liquid sample receiving member can permeate sequentially through the conjugate pad and the first carrier strip; (4) an adsorbent pad disposed upon said dry porous strip such that said absorbent pad is positioned opposite said conjugate pad and such that said reaction site lies in-between said conjugate pad and said first absorbent pad; and
a housing capable of holding at least one test strip wherein said housing is further capable of applying pressure to said test strip components, said housing comprising the following separate parts: (1) a substrate; (2) at least one well within said substrate capable of encasing a test strip; (3) a cap for covering said substrate, said cap capable of exerting pressure upon said test strip components when affixed to said substrate to ensure flowability of sample throughout said test strip components.

28. The kit of claim 27 wherein said analyte is selected from the group consisting of monoclonal free lambda light chain and monoclonal free kappa light chain.

29. A kit for determining the presence of Bence Jones protein in urine sample comprising;
at least one device of claim one, wherein said device further comprises a housing suitable for holding at least one devices of claim 1 and applying pressure to said device, said housing comprising the following separate parts: (1) a substrate; (2) at least two wells within said substrate capable of encasing at least one device of claim 1; (3) a cap for covering said substrate, said cap capable of exerting pressure upon said device of claim 1 when said cap is affixed to said substrate to ensure lateral flowability of sample throughout said device. Express Mail No. EL 889 837 939 US
